(19) United States
(12) Patent Application Publication
(10) Pub. No.: US 2020/0158652 A1
(43) Pub. Date: May 21, 2020

(54) COLORIMETRIC DETECTION OF ENERGETIC MATERIALS

(71) Applicant: Lawrence Livermore National Security, LLC, Livermore, CA (US)

(72) Inventors: John Reynolds, San Ramon, CA (US); Lara Leininger, Livermore, CA (US); Ana Racoveanu, Livermore, CA (US)

(21) Appl. No.: 16/194,174

(22) Filed: Nov. 16, 2018

(51) Int. Cl. G01N 21/78; G01N 1/02; G01N 33/22; B01L 3/00

(52) U.S. Cl. CPC G01N 21/78; G01N 1/02; G01N 33/227; G01N 2001/028; B01L 2200/16; G01N 2001/027; B01L 3/5023

(57) ABSTRACT

Exemplary systems for detecting energetic materials include: a sample wipe comprising a fibrous substrate configured to absorb and/or adhere to the energetic material; means for applying at least a first reagent configured to produce a visible color upon reaction with the energetic material to the sample wipe; means for applying at least a second reagent configured to produce a second visible color upon reaction with the energetic material to the sample wipe; and means for applying at least one solvent configured to solvate the energetic material to the sample wipe. Sample wipes may also be used independently to detect energetic materials, and include: a fibrous substrate configured to absorb/adhere to an energetic material; and a solvent configured to solvate the energetic material. Methods for detecting presence of energetic materials in a sample are also disclosed.

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD OF THE INVENTION

The present invention relates to detection of energetic materials, and more particularly, this invention relates to detection of energetic materials typically undetectable using conventional colorimetric detection techniques, including but not limited to insoluble explosive materials.

### BACKGROUND

Detection of energetic materials such as explosives remains an important aspect of national security and military operations across the world, especially in theatres where improvised explosive devices are deployed.

Colorimetric techniques are the gold-standard conventional approach to detection of energetic materials such as explosives. These methods are generally the fastest, least expensive and most overall comprehensive methods for detecting explosives in field situations. However, these methods do not work well with certain energetic materials, particularly insoluble explosives such as tri-amino-tri-nitro-benzene (TATB), especially when the energetic molecule is in a polymer-bound formulation.

There is therefore a need for fast, reliable, portable and inexpensive techniques to detect the presence of insoluble explosives, including polymer-bound explosives, and the like, using colorimetric techniques typically incapable of detecting such energetic materials.

### SUMMARY

According to one embodiment, a system for detecting presence of insoluble explosives in a sample includes: a sample wipe comprising a fibrous substrate configured to absorb and/or adhere to the energetic material; means for applying at least a first reagent configured to produce a visible color upon reaction with the energetic material to the sample wipe; means for applying at least a second reagent configured to produce a second visible color upon reaction with the energetic material to the sample wipe; and means for applying at least one solvent configured to solvate the energetic material to the sample wipe.

According to another embodiment, a sample wipe includes: a fibrous substrate configured to absorb/adhere to an energetic material; and a solvent configured to solvate the energetic material.

According to yet another embodiment, a method of detecting presence of insoluble explosives in a sample includes: exposing a sample wipe, having at least one solvent that solvates an energetic material thereon, to a test material or a test environment; exposing the sample wipe to at least a first reagent; and determining whether the sample wipe exhibits a change in color in response to exposure of the sample wipe to the first reagent. A change in color of the sample wipe is indicative of a presence of the energetic material.

Other aspects and advantages of the present invention will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, as well as the preferred mode of use, reference should be made to the following detailed description read in conjunction with the accompanying drawings.

FIGS. 1A-1B are simplified schematics of a conventional system for colorimetric detection of energetic materials.

FIG. 2 is a simplified schematic of an inventive system for colorimetric detection of insoluble explosives, according to one embodiment of the presently disclosed inventive concepts.

FIG. 3 is a flowchart of a method, according to another embodiment of the presently disclosed inventive concepts.

FIGS. 4A-4C are schematic representations of conventional Easy Livermore Inspection Test for Explosives (ELITE) kits after exposure to insoluble explosive materials polymer-bound explosive 9502 (PBX 9502) (FIGS. 4A-4B) or triaminotrinitrobenzene (TATB) (FIG. 4C), and the resulting lack of any colorimetric change. The schematics were copied from actual photographs of the ELITE kits.

FIGS. 5A-5C are schematic representations of inventive colorimetric detection kits including a solvent, after exposure to TATB, and the resulting colorimetric change. The schematics were copied from actual photographs of the inventive colorimetric detection kits.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

As utilized herein, the term “about” refers to a given value, ±10% of the given value.

The term “energetic material” as utilized herein shall be understood as referring to materials with sufficient energetic density to produce a self-propagating exothermic chemical reaction upon initiation thereof. Preferably, energetic materials refer to explosive materials, but in various embodiments may include other energetic materials such as thermites, intermetallic compounds, etc. as would be understood by a person having ordinary skill in the art upon reading the present descriptions.

The term “insoluble explosives” shall be understood as referring to energetic materials, which, by design or by virtue of the chemical structure, are substantially insoluble in conventional solvents used in conventional colorimetric detection techniques. For example, in preferred embodiments TATB-based explosives are characterized by a solubility of less than 1 ppm in conventional solvents used for colorimetric detection, such as methanol, ethanol, etc. as would be understood by a person having ordinary skill in the art upon reading the present disclosure. Exemplary insoluble explosives, in accordance with various embodiments of the presently disclosed inventive concepts, include, but are not limited to: polymer-bound explosives (PBX) such as PBX-9502, triaminotrinitrobenzene (TATB), T2, and any combination thereof. PBX-9502 is essentially 95% TATB mixed with an appropriate polymer (e.g. Kel-F 800); while T2 is essentially 97% TATB mixed with an appropriate binder.

The following description discloses several preferred embodiments of systems and techniques for the detection of energetic materials, particularly insoluble explosives, using colorimetric techniques and/or related systems and methods.

In one general embodiment, a system for detecting presence of insoluble explosives in a sample includes: a sample wipe comprising a fibrous substrate configured to absorb and/or adhere to the energetic material; means for applying at least a first reagent configured to produce a visible color upon reaction with the energetic material to the sample wipe; means for applying at least a second reagent configured to produce a second visible color upon reaction with the energetic material to the sample wipe; and means for applying at least one solvent configured to solvate the energetic material to the sample wipe.

According to another general embodiment, a sample wipe, includes: a fibrous substrate configured to absorb/adhere to an energetic material; and a solvent configured to solvate the energetic material.

According to yet another general embodiment, a method of detecting presence of insoluble explosives in a sample includes: exposing a sample wipe, having at least one solvent that solvates an energetic material thereon, to a test material or a test environment; exposing the sample wipe to at least a first reagent; and determining whether the sample wipe exhibits a change in color in response to exposure of the sample wipe to the first reagent. A change in color of the sample wipe is indicative of a presence of the energetic material.

The presently disclosed inventive concepts are directed to colorimetric detection of energetic materials. While a primary advantage of the inventive concepts described herein is ability to reliably detect presence of insoluble explosives, such as polymer-bound explosives, and the like (which remain undetectable using conventional colorimetric techniques), it shall be understood that the systems and methods described herein are equally applicable to colorimetric detection of energetic materials that are also capable of detection using conventional colorimetric techniques (such as trinitrotoluene (TNT)). Indeed, employing the inventive concepts presented herein to the colorimetric detection of such materials, in various embodiments, yields a lower detection limit (increased sensitivity). Accordingly, employing the presently described inventive concepts may, depending on the chemistry of the energetic material in question, improve the detection limit for that material. Moreover, the enhanced solubility offered by use of specific solvents configured to solvate insoluble explosives may, in some approaches, enhance the rate of dissolution of the insoluble explosive in the solvent. Accordingly, in practical applications, such as field detection event, time dependent detection is enhanced according to some embodiments.

As mentioned above, colorimetric detection of energetic materials is a technique generally known to be suitable for detection of many materials, but generally not for insoluble explosives such as described herein. Accordingly, the presently disclosed inventive concepts may be considered an extension, expansion, or improvement upon the conventional colorimetric techniques, which extends detection capability to include insoluble explosives.

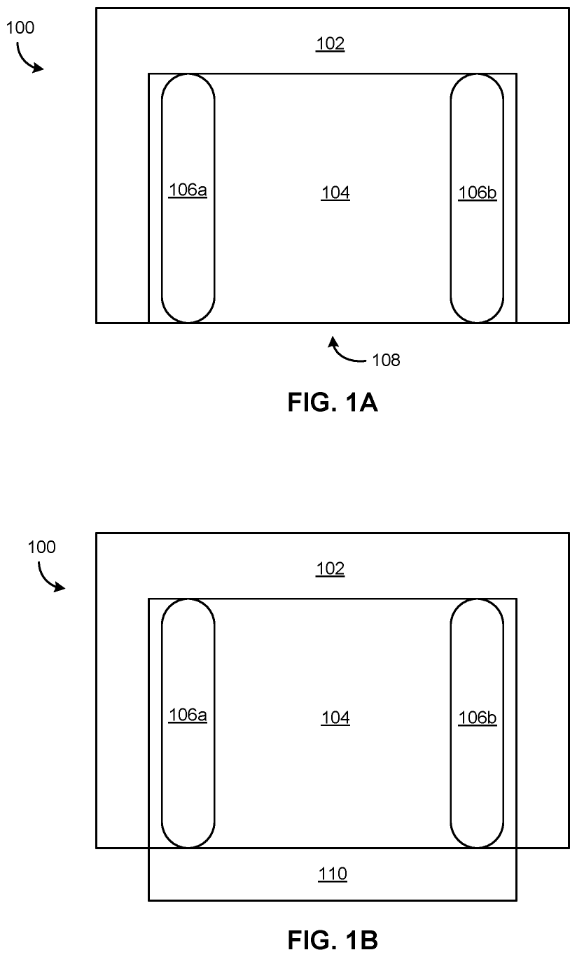

Referring now to FIG. 1A-1B, a simplified schematic of a conventional system 100 for colorimetric detection of energetic materials is shown. The system 100 substantially represents an Easy Livermore Inspection Test for Explosives (ELITE) kit, as described in further detail below.

With continuing reference to FIGS. 1A and 1B, the system 100 consists of a reaction chamber 104 surrounded by an enclosure 102. The reaction chamber 104 may be provided in the form of a central void formed in the enclosure 102 and spatially configured to receive a sample wipe 110 via a slot or port 108 formed in one side of the enclosure 102. The sample chamber 104 and enclosure 102 are also preferably configured so as to mitigate or prevent contamination of the sample by dust, debris, or other materials present in the operating environment.

The system 100 also includes two reagent chambers 106a, 106b positioned at opposite sides of the reaction chamber 104. The reagent chambers 106a, 106b contain reagents for the colorimetric detection of energetic materials, namely a first reagent (also referred to as “reagent A”) reactive to aromatic explosive compounds; and/or a second reagent (also referred to as “reagent B”) reactive to nitro-aliphatic-based explosives and nitramine-based explosives. For all reagents, the reaction, if any occurs, produces a visible color indicating presence of the corresponding type of energetic material.

Optionally, the system 100 may include a heat source such as a heating element (not shown) configured to heat the system, or at least the sample wipe 110 when placed in the reaction chamber 104. Applying heat during testing may improve detection of certain types of energetic materials, especially nitro-aliphatic compounds and/or nitramine-based compounds.

Returning now to the conventional ELITE kit mentioned above, this kit uses colorimetric chemistry, which displays positive for energetic materials by showing color. The object to be sampled is sample wiped with the applicator provided by the kit. In various inventive approaches, there are two indicator steps. The first employs Meisenheimer reaction chemistry that produces a colored compound with aromatic explosives, such as TNT. The second employs Greiss reaction chemistry to produce a colored compound with nitro-aliphatic or nitramine explosives, such as RDX or HMX. The appearance of the colored compound means a positive reaction and therefore the presence of an explosive.

The ELITE kit is a solid phase-liquid phase reaction system. A dry sample wipe is used to collect dry residue. The agents, in mostly aqueous alcohol such as methanol, ethanol, etc. are applied to essentially solid phase materials. The ELITE kit functions by applying a provided sample wipe and contacting the source in question. The sample wipe is then placed back in the ELITE kit and one or more reagents are applied to the sample wipe.

Reagent A causes the first type of chemistry described above to proceed. The reagent is stored in a small glass ampoule (e.g. ampoule 106a) next to the edge of the sample wipe that is broken by the thumb or forefinger. The reagent then migrates across the sample wipe. If there is nitro-aromatic explosive present, then the reagent reacts with it and changes the color on the surface of the sample wipe. If there is no indication of color change, the sample is considered free from nitro aromatic explosives.

The system is then heated and Reagent B, which resides on the other edge of the sample wipe (e.g. in ampoule 106b), is applied in the same manner. If the nitramine is present, then the reagent reacts with it and changes the color on the surface of the sample wipe. If there is no evidence of color, the sample is considered free from aliphatic explosives.

The ELITE kit was developed for the military to determine if explosives were present in rogue operations, such as suicide bombing and improvised explosive devices. At the time, the explosives of concern were standard munition-type explosives, such as found in 155 mm munitions, and improvised explosive mixtures, such as ammonium nitrate mixtures. For security reasons in the theater, these were very important targets. The ELITE kit that was developed has excellent sensitivity for these explosives and was largely deployed.

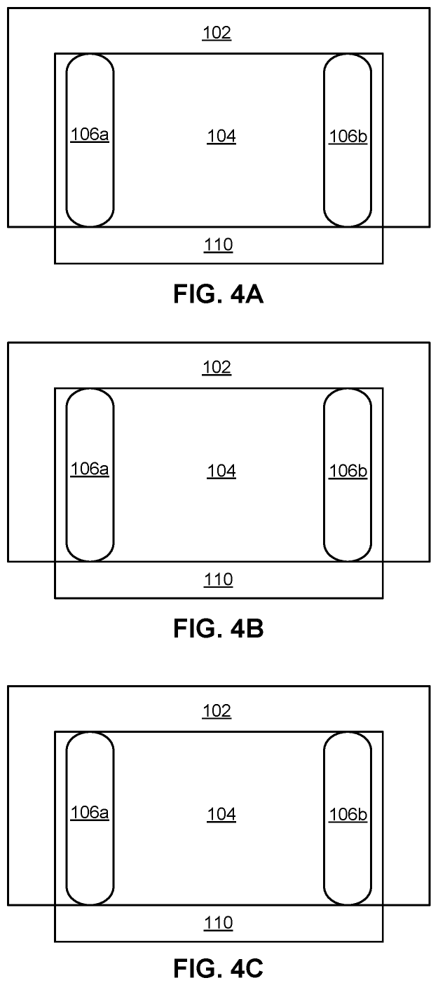

However, when conventional systems such as the ELITE kit are applied to insoluble explosive samples, e.g. PBX-9502, TATB and/or T2 formulations, positive indication remains elusive, and non-conclusive, as shown and described in greater detail with reference to FIGS. 4A-4C below.

This lack of sensitivity to TATB and similar insoluble explosives was surprising, and unexpected. First, as a nitro-aromatic explosive, TATB should have formed a colored complex with Reagent A of the ELITE kit, making a Meisenheimer complex, which is highly colored, usually red or purple. Second, TATB also has 3 nitro groups, so, under the right conditions, should form a pink complex with Reagent B of the ELITE kit, submitting to Greiss reagent chemistry. However, in practice, the expected chemistry did not occur and visual indication of the energetic materials' presence was not reproducible.

Accordingly, the inventors set out to develop new techniques for colorimetric detection of energetic materials, capable of detecting insoluble explosives such as TATB and the like in a fast, reliable, portable and inexpensive manner. The following descriptions set forth the various features of the invention with exemplary reference to TATB as the energetic material to be detected. However, it should be understood that the inventive concepts described herein are equally applicable to other insoluble explosives and even to energetic materials capable of detection using a conventional system and/or colorimetric techniques. Indeed, even for energetic materials capable of detection using a conventional system and/or colorimetric techniques, implementing the inventive concepts of the present disclosure can improve detection by effectively lowering detection limit and/or sensitivity of the system to the energetic material.

In essence, and as a surprise given the particularly low solubility of many insoluble explosives, facilitating detection of such insoluble explosives is accomplished via application of a solvent to the sample. The solvent is preferably a solvent of the energetic material sought for detection, even if only to a slight degree (e.g., due to the energetic material's inherent resistance to solvation by a wide variety of solvents). The solvent is preferably a solvent of the energetic material sought for detection, even if only to a slight degree (e.g., due to the energetic material's inherent resistance to solvation by a wide variety of solvents) to yield limits of detection (LOD) of about 5 nanograms, and preferably more. However, as will be appreciated by persons having ordinary skill in the art, LOD varies with each explosive.

In various approaches, the sample wipe may be directly exposed to the solvent immediately before sampling, and/or the sample wipe can be pre-exposed to the solvent, placed in a sealed container and taken to the field. Accordingly, in one approach the sample wipe may be provided in a manner ready for immediate use upon opening the sealed container to access the wipe.

With continuing reference to the sample wipe and solvent combination, more sophisticated embodiments include, but are not limited to, microencapsulation and impregnation of thixotropic solids such as Cab-O-Sil with the selected solvent.

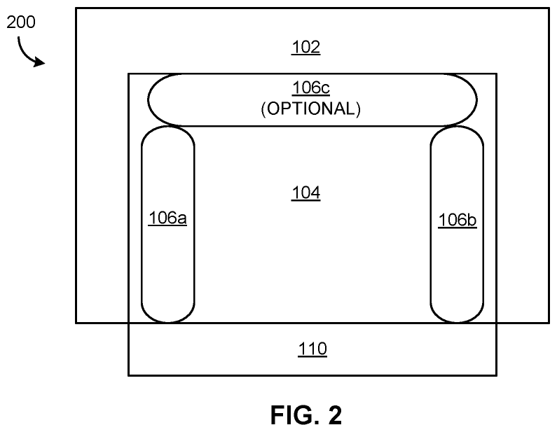

In any case, the solvent system is preferably applied throughout the sample wipe in a pre-treatment process before sampling. The action of swiping and breaking of the capsule liberates the solvents from the solids, which is wicked into the sample wipe, enhancing the detection as demonstrated above. These systems have the advantage of decreasing exposure of the operator of the kit to the solvent. Other methods include additional ampoules that contain the solvent that are activated before use of the sample wipe, but are also portable with the kit. The ampoule is then broken, e.g. with a thumb or forefinger, prior to using the sample wipe, e.g. using a system 200 such as shown in FIG. 2 and described in greater detail below.

Again, in the exemplary context of detecting TATB, suitable solvents include dimethyl formamide (DMF), dimethyl sulfoxide (DMSO) and hexamethyl phosphoramide (HMPA), which may be employed singly or in any combination in various embodiments.

Referring now to FIG. 2, an exemplary system 200 for colorimetric detection of energetic materials is shown, according to one embodiment of the presently described inventive concepts. Although in various approaches insoluble explosives may be detected even using a conventional system such as shown in FIGS. 1A-1B, in preferred approaches an inventive system such as shown in FIG. 2 is employed.

The inventive system 200 is substantially similar, in one embodiment, to the structure of the ELITE kit, e.g. as shown and described above with reference to FIGS. 1A-1B. However, system 200 optionally includes a third reagent chamber 106c preferably containing the solvent and configured to apply and/or facilitate application of the solvent to a sample wipe 110 placed in the reaction chamber 104. It should be understood that the third reagent chamber 106c is optional, as solvent may be applied to the sample wipe 110 in any suitable manner described herein, and need not be applied via reagent chamber 106c or even using a system such as shown in FIG. 2. For instance, in alternative approaches a sample wipe may be exposed to the reagents and/or solvents using any suitable means for applying a liquid to a solid, such as soaking, spraying, wicking, etc. as would be understood by a person having ordinary skill in the art upon reading the present disclosure.

The system 200 also includes an enclosure 102 having a slot 108 formed therein to allow placement of a sample wipe 110 in the reaction chamber 104, as well as reagent chambers 106a and 106b disposed in and/or fluidically coupled to the reaction chamber 104.

Of course, those having ordinary skill in the art will appreciate that the foregoing components and corresponding features of system 200 may be employed in any suitable combination or permutation thereof. Moreover, materials and components that skilled artisans would consider functionally and/or structurally equivalent to those expressly described above in connection with system 200 may be employed without departing from the scope of the presently described inventive concepts, according to various embodiments.

For example, in various embodiments the inventive systems described herein require only the sample wipe, and means for applying appropriate solvent(s) and/or reagent(s) to the sample wipe before, during, or after exposing the sample wipe to a test sample suspected of including insoluble explosives. The sample wipe preferably is or comprises a fibrous substrate configured to absorb and/or otherwise adhere to or attract a sample of interest, most preferably high explosive materials including but not limited to insoluble explosives. Suitable means for applying the appropriate reagent(s) and/or solvent(s) include, in several illustrative embodiments, one or more reagent chambers as described hereinabove with reference to FIG. 2, a dropper, a spray bottle, an aerosol dispenser, a pipette, a burette, a syringe, a tub, tank or other enclosure into which the sample wipe may be submerged/placed, break-up of microencapsulation, breaking an ampoule, or any combination thereof, and equivalents thereof that would be appreciated by persons having ordinary skill in the art upon reading the present specification.

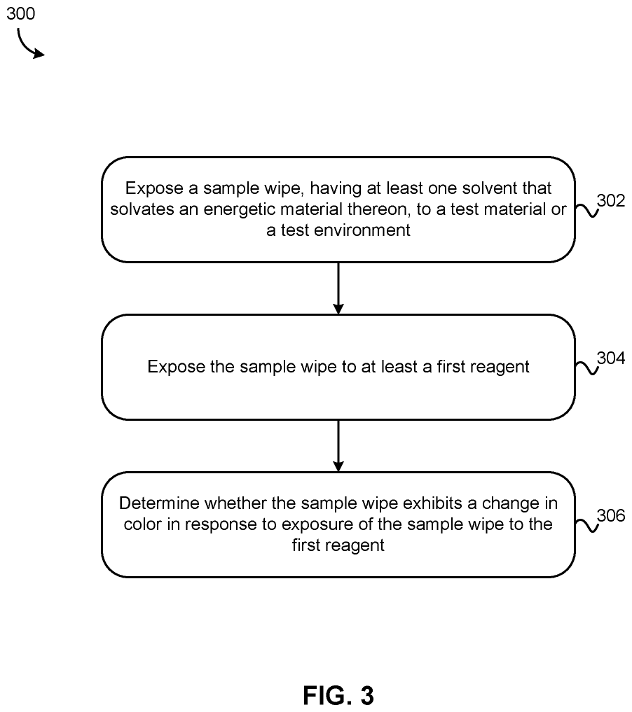

Turning now to FIG. 3, a flowchart of a method 300 for detecting an energetic material is shown, according to one embodiment. Those having ordinary skill in the art will appreciate the method 300 may be performed using any of the systems described herein, including those shown in FIGS. 1A-1B and 2, in various approaches, though system 200 as shown in FIG. 2 above is preferred for at least the reasons stated hereinabove. Moreover, the method 300 may include more or less operations than those shown in FIG. 3, as well as different and/or additional features, limitations, etc. than shown in FIG. 3, all without departing from the scope of the inventive concepts described herein.

As shown in FIG. 3, method 300 includes operation 302, where a sample wipe having a solvent applied thereto, included therewith, or otherwise present in the sample wipe, is exposed to a test material and/or test environment. The solvent solvates the energetic material sought for detection, and is chosen based on solubility of the energetic material sought for detection therein, with higher solubility being preferred. Those having ordinary skill in the art will appreciate that many insoluble explosives and other energetic materials are non-reactive (e.g. TATB) and therefore may have solubilities as low as 0.5 g/L or less in the chosen solvent. Regardless, this solubility is superior to the solubility of the energetic material/insoluble explosive in other solvents.

Although the sample wipe may be exposed to the solvent in any suitable manner, preferably the solvent is applied via prepackaging the sample wipe in a container containing the solvent, and sealing the container until ready for use. Alternatively, the sample wipe may be exposed to the solvent by placing the sample wipe in an analysis apparatus (e.g. as shown in FIG. 2), and a reagent chamber (e.g. optional reagent chamber 106c) may be ruptured to release the solvent, which is wicked up by the sample wipe.

Of course, any suitable technique of exposing the sample wipe to the solvent as described herein, and equivalents thereof that would be appreciated by a skilled artisan upon reading these descriptions, may be employed without departing from the scope of the inventive concepts presently disclosed. For example, in various approaches the solvent and/or various reagents may be applied to the sample wipe by dropping droplets of the reagent/solvent, spraying the reagent/solvent onto the sample wipe, partially or wholly submerging the sample wipe in a container containing the reagent and/or solvent, or any other appropriate technique that would be appreciated by a person having ordinary skill in the art upon reviewing the present application.

Similarly, the solvent may be any solvent suitable for solvating the energetic material sought for detection, including but not limited to dimethyl formamide (DMF), dimethyl sulfoxide (DMSO), and/or hexamethyl phosphoramide (HMPA) in any combination or permutation.

The sample wipe is exposed to a first reagent in operation 304. While the apparatus as shown in FIGS. 1A-1B and 2 each are suitable for use in the context of method 300, other techniques for applying reagent and/or solvent to the sample wipe may be employed in the context of operation 304 without departing from the scope of the inventive concepts presented herein.

In operation 304 the first reagent is preferably Reagent A as described above, and is exposed to the reagent using any suitable technique, e.g., by rupturing at least a first reagent chamber containing the first reagent, dropping a predetermined volume of the first reagent onto the sample wipe, partially or wholly submerging the sample wipe in a container containing the first reagent, etc. The amount of first reagent provided to the sample wipe preferably is sufficient to expose substantially all (e.g. at least 75% of a volume of the sample wipe) of the sample wipe to the reagent. A change in color of the sample wipe in response to exposure to the first reagent is indicative of presence of an energetic material, preferably an insoluble aromatic explosive compound.

Of course, in further embodiments of method 300, the sample wipe may be sequentially exposed to additional reagents (optionally contained in additional reagent chambers or other delivery mechanisms), e.g. a second reagent and/or a solvent, which may be contained in second and/or third chambers respectively (e.g. reagent/solvent chambers 106b/106c, respectively).

Accordingly in some embodiments method 300 may, but need not, include exposing the sample wipe to at least a second reagent (e.g. Reagent B), again optionally by rupturing at least a second reagent chamber containing the second reagent or using any other delivery mechanism/technique described herein; determining whether the sample wipe exhibits a change in color in response to exposure of the sample wipe to the second reagent; and in response to determining the sample wipe exhibits the change in color, determining a presence and a concentration of the energetic material. In other words, a change in color of the sample wipe upon or following exposure thereof to reagent B is indicative of presence of an insoluble explosive, e.g. a nitro-aliphatic-based explosive compound and/or a nitramine-based explosive compound.

Similarly, in some embodiments method 300 may, but need not, include exposing the sample wipe to a solvent by rupturing at least a third chamber containing the solvent. The third chamber may be ruptured prior to or after exposing the sample wipe to the energetic material, in various approaches.

Of course, Reagents A and B may be employed in any order, combination, or permutation without departing from the scope of the presently disclosed inventive concepts. Only one of the reagents may be applied, or multiple reagents may be applied, in various approaches. Similarly, the Reagents A and B may interchangeably comprise a Greiss reagent, a reagent configured to form a Meisenheimer complex with the energetic material upon reaction therewith, or any other suitable type of reagent for the colorimetric detection of energetic materials of a particular type.

With continuing reference to FIG. 3, method 300 includes operation 306, wherein a determination is made as to whether the sample wipe exhibits a change in color, in response to exposure of the sample wipe to the first reagent. As noted above, a change in color is generally indicative of presence of the energetic material, especially where the change is drastic and/or persists for 24 hours or more (e.g. in the case of nitro-aliphatic and/or nitramine-based compounds reacting with a Greiss reagent). A “drastic” change, as referenced herein, shall be understood as a change in color significant enough for the human eye to detect without ambiguity.

Optionally, method 300 may also involve determining a concentration of the energetic material in the sample wipe, using any suitable technique known in the art.

Experimental Results

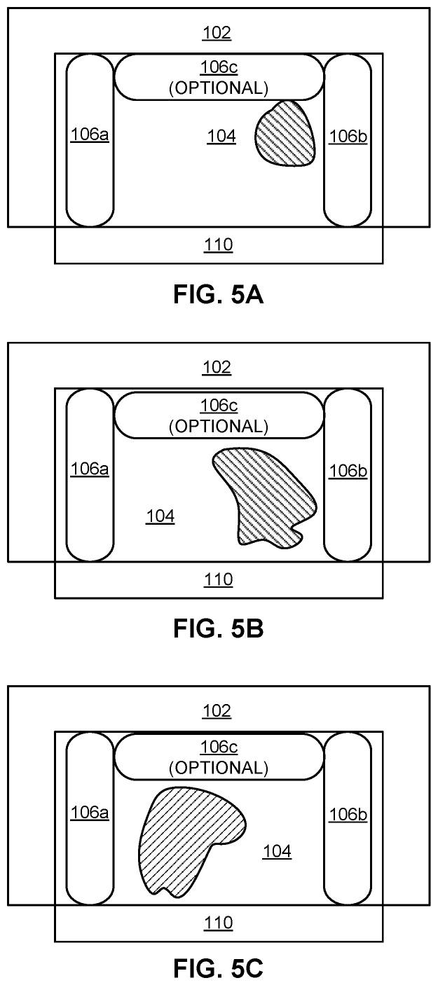

Again, using TATB as an exemplary insoluble explosive material, along with polymer bound explosive 9502 (PBX 9502), results of attempting colorimetric detection without (FIGS. 4A-4C) and with (FIGS. 5A-5C) pre-treating the sample wipe with solvent are shown, according to several embodiments.

In FIGS. 4A and 4B, the sample is PBX 9502, while in FIG. 4C the sample is a single crystal TATB formulation. Note, these samples are materials that have 90+% TATB, not samples of trace environmental contamination, so a positive should be intense and easily recognized. The slight color of the PBX 9502 samples could be justified by the composition of the material containing a well-dispersed 5% Kel-F polymer. However, the single crystal TATB shows no color, which is a clear indication that the kit is not functioning.

By comparison, FIGS. 5A-5C show several exemplary schematics, copied from actual photographs, of sample wipes exposed to TATB, with different solvent and/or reagents applied for detection of the TATB. FIG. 5A shows a sample wipe pretreated in DMF solvent followed by application of Reagent A only. FIG. 5B shows a sample wipe pretreated in DMSO solvent, followed by application of Reagent A only. FIG. 5C shows the sample wipe in FIG. 5A, after the additional application of Reagent B (Griess). The corresponding photograph was taken approximately 24 hours after the application of the reagents. In this case, the Griess reagent sustains the color longer, which is an advantage in record keeping. The DMSO pretreated sample was not exposed to Reagent B and did not retain the color overnight (not shown), typical of the Meisenheimer complex.

Both the DMF and DMSO pretreated sample wipes exhibit enhanced response to the detection reagents. There is no ambiguity about the positive character of the result. Comparing this result to that of FIG. 4C for the single crystal TATB, shows a dramatic improvement of detection of TATB.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. A system for colorimetric detection of an energetic material, the system comprising:
a sample wipe comprising a fibrous substrate configured to absorb and/or adhere to the energetic material;
means for applying at least a first reagent configured to produce a visible color upon reaction with the energetic material to the sample wipe;
means for applying at least a second reagent configured to produce a second visible color upon reaction with the energetic material to the sample wipe; and
means for applying at least one solvent configured to solvate the energetic material to the sample wipe.

2. The system as recited in claim 1, wherein the first reagent is configured to form a Meisenheimer complex with the energetic material upon contact therewith.

3. The system as recited in claim 1, wherein the second reagent is a Greiss reagent.

4. The system as recited in claim 1, wherein the sample wipe is pre-soaked with the at least one solvent.

5. The system as recited in claim 1, wherein each solvent is independently selected from the group consisting of: dimethyl formamide (DMF), dimethyl sulfoxide (DMSO), and hexamethyl phosphoramide (HMPA).

6. The system as recited in claim 1, wherein the energetic material comprises an insoluble explosive.

7. The system as recited in claim 6, wherein the insoluble explosive comprises one or more materials selected from the group consisting of: triaminotrinitrobenzene (TATB), PBX-9502, and T2.

8. A sample wipe, comprising:
a fibrous substrate configured to absorb/adhere to an energetic material; and
a solvent configured to solvate the energetic material.

9. The sample wipe as recited in claim 8, wherein the sample wipe is enclosed in a sealed container comprising the solvent.

10. The sample wipe as recited in claim 8, wherein the solvent comprises one or more solvents selected from the group consisting of: dimethyl formamide (DMF), dimethyl sulfoxide (DMSO), and hexamethyl phosphoramide (HMPA).

11. The sample wipe as recited in claim 8, wherein the energetic material comprises an insoluble explosive selected from the group consisting of: triaminotrinitrobenzene (TATB), PBX-9502, and T2.

12. A method of detecting an energetic material, the method comprising:
exposing a sample wipe, having at least one solvent that solvates an energetic material thereon, to a test material or a test environment;
exposing the sample wipe to at least a first reagent; and
determining whether the sample wipe exhibits a change in color in response to exposure of the sample wipe to the first reagent; and
wherein the change in color of the sample wipe is indicative of a presence of the energetic material.

13. The method as recited in claim 12, wherein the first reagent forms a Meisenheimer complex with the energetic material upon reaction therewith.

14. The method as recited in claim 12, further comprising
exposing the sample wipe to at least a second reagent; and
determining whether the sample wipe exhibits a change in color in response to exposure of the sample wipe to the second reagent; and
wherein the change in color of the sample wipe is indicative of a presence of the energetic material.

15. The method as recited in claim 14, wherein the second reagent forms a Griess complex with the energetic material upon reaction therewith.

16. The method as recited in claim 12, wherein the at least one solvent comprises one or more compounds selected from the group consisting of: dimethyl formamide (DMF), dimethyl sulfoxide (DMSO), and hexamethyl phosphoramide (HMPA).

17. The method as recited in claim 12, wherein the energetic material comprises an insoluble explosive.

18. The method as recited in claim 17, wherein the insoluble explosive comprises one or more materials selected from the group consisting of: triaminotrinitrobenzene (TATB), PBX-9502, and T2.

19. The method as recited in claim 12, further comprising applying the at least one solvent to the sample wipe using a medicine dropper and/or a spray bottle.

20. The method as recited in claim 12, wherein the sample wipe is provided in a pre-packaged form including the sample wipe and the solvent.
